(19) United States
(12) Patent Application Publication
(10) Pub. No.: US 2014/0017673 A1
(43) Pub. Date: Jan. 16, 2014

(54) METHOD FOR ENUMERATING EUKARYOTIC CELL MICRONUCLEI WITH AN EMPHASIS ON SIMULTANEOUSLY ACQUIRING CYTOTOXICITY AND MODE OF ACTION INFORMATION

(71) Applicant: LITRON LABORATORIES, LTD., Rochester, NY (US)

(72) Inventors: Stephen D. Dertinger, Webster, NY (US); Steven M. Bryce, Rochester, NY (US)

(73) Assignee: LITRON LABORATORIES, LTD., Rochester, NY (US)

(21) Appl. No.: 13/791,912

(22) Filed: Mar. 9, 2013

Related U.S. Application Data
(60) Provisional application No. 61/670,394, filed on Jul. 11, 2012.

(51) Int. Cl. G01N 33/68; C12Q 1/68

(52) U.S. Cl. CPC G01N 33/6875; C12Q 1/68
USPC 435/6.1

(57) ABSTRACT

The present invention relates a method for the enumeration of eukaryotic cell micronuclei, while simultaneously acquiring cytotoxicity and mode of action information. The method utilizes differential labeling of chromatin from dead and dying cells to distinguish the chromatin from micronuclei, nuclei, and metaphase chromosomes, and differential labeling of metaphase events to provide additional information regarding cytotoxicity and genotoxic modes of action. Counting of micronuclei events relative to the number of nuclei and quantifying perturbations to the proportion of metaphase events can be used to assess the DNA-damaging potential of a chemical agent, the DNA-damaging potential of a physical agent, the effects of an agent which can modify endogenously-induced DNA damage, the effects of an agent which can modify exogenously-induced DNA damage, and genotoxic mode of action.

This application claims the priority benefit of U.S. Provisional Patent Application Ser. No. 61/670,394, filed Jul. 11, 2012, which is hereby incorporated by reference in its entirety.

### FIELD OF THE INVENTION

This invention relates to a method for enumerating eukaryotic cell micronuclei while simultaneously acquiring information that characterizes treatment-related cytotoxicity and, in the case of micronucleus induction, provides evidence for whether the genotoxic activity is the result of an aneugenic or clastogenic mode of action.

### BACKGROUND OF THE INVENTION

The induction of DNA damage and the resulting sequelae of mutations and chromosomal rearrangements are primary mechanisms by which cancers arise. These types of events have also been implicated in diseases such as atherosclerosis, processes such as aging, and the development of birth defects such as Down syndrome. Therefore, there is an important need for sensitive methods which are capable of identifying chemical or physical agents that can alter DNA. Given the tremendous cost of long-term chronic studies such as 2-year carcinogenicity tests, short- and medium-term systems for predicting DNA damage potential continue to play a vital role in tumorigenic agent identification. In fact, the need for short-term tests that have a high throughput capacity has never been greater. Advances in molecular biology and combinatorial chemistry have provided large numbers of potential targets and many novel compounds that may be useful for treating or preventing disease. However, before such agents can be used in clinical practice, acceptable toxicity/safety profiles must be demonstrated. In the area of environmental health and safety, many natural and industrially manufactured compounds and formulations have not been adequately evaluated for toxicity. In both arenas, traditional toxicity evaluations are labor intensive and require extensive use of in vivo assays. This situation offers opportunities for methods that are able to quickly and inexpensively determine toxicological profiles of potential therapeutic drugs and environmental agents.

Micronuclei (MN) are formed upon cell division in cells with DNA double-strand break(s) or dysfunctional mitotic spindle apparatus. Based on this detailed understanding of micronuclei origin, the rodent-based micronucleus test has become the most widely utilized in vivo system for evaluating the clastogenic and aneugenic potential of chemicals (Heddle, “A Rapid In Vivo Test for Chromosome Damage,” Mutat. Res. 18:187-190 (1973); Schmid, “The Micronucleus Test,” Mutat. Res. 31:9-15 (1975); Hayashi et al., “In Vivo Rodent Erythrocyte Micronucleus Assay. II. Some Aspects of Protocol Design Including Repeated Treatments, Integration With Toxicity Testing, and Automated Scoring,” Environ. Mol. Mutagen. 35:234-252 (2000)). These rodent-based tests are most typically performed as erythrocyte-based assays. Since erythroblast precursors are a rapidly dividing cell population, and their nucleus is expelled a few hours after the last mitosis, micronucleus-associated chromatin is particularly simple to detect in reticulocytes and normochromatic erythrocytes given appropriate staining (e.g., acridine orange) (Hayashi et al., “An Application of Acridine Orange Fluorescent Staining to the Micronucleus Test,” Mutat. Res. 120:241-247 (1983)).

One of the short-term test systems that is becoming widely used to screen drug candidates and other chemicals for genotoxic activity is the in vitro micronucleus test. Analogous to the way in vivo erythrocyte-based micronucleus tests have become more common than in vivo chromosome aberration analyses, a consensus has been reached whereby the in vitro micronucleus assays is considered a reliable substitution for in vitro chromosome aberration studies. While both endpoints are capable of detecting agents that cause cytogenetic damage, in vitro micronucleus formation is technically easier to perform and score.

Given the growing enthusiasm for the in vitro micronucleus endpoint, numerous efforts to automate the scoring phase of the technique have been described in the literature—methods based on image analysis, laser scanning cytometry, and flow cytometry have all been reported (Nüsse et al., “Flow Cytometric Analysis of Micronuclei Found in Cells After Irradiation,” Cytometry 5:20-25 (1984); Schreiber et al., “An Automated Flow Cytometric Micronucleus Assay for Human Lymphocytes,” Int. J. Radiat. Biol. 62:695-709 (1992); Schreiber et al., “Multiparametric Flow Cytometric Analysis of Radiation-Induced Micronuclei in Mammalian Cell Cultures,” Cytometry 13:90-102 (1992); Vral et al., “The In Vitro CytoKinesis-Block Micronucleus Assay: A Detailed Description of an Improved Slide Preparation Technique for the Automated Detection of Micronuclei in Human Lymphocytes,” Mutagenesis 9:439-443 (1994); Verhaegen et al., “Scoring of Radiation-Induced Micronuclei in Cytokineses-Blocked Human Lymphocytes by Automated Image Analysis,” Cytometry 17:119-127 (1994); Böcker et al., “Image Processing Algorithms for the Automated Micronucleus Assay in Binucleated Human Lymphocytes,” Cytometry 19:283-294 (1995); Wessels et al., “Flow cytometric Detection of Micronuclei by Combined Staining of DNA and Membranes,” Cytometry 19:201-208 (1995); Viaggi et al., “Flow Cytometric Analysis of Micronuclei in the CD2+ Subpopulation of Human Lymphocytes Enriched by Magnetic Separation,”Int. J. Radiat. Biol. 67:193-202 (1995); Nüsse et al., “Flow Cytometric Analysis of Micronuclei In Cell Cultures and Human Lymphocytes: Advantages and Disadvantages,” Mutat. Res. 392:109-115 (1997); Roman et al., “Evaluation of a New Procedure for the Flow Cytometric Analysis of In Vitro, Chemically Induced Micronuclei in V79 Cells,” Environ. Molec. Mutagen. 32:387-396 (1998)).

The most established technique for high throughput in vitro MN scoring, both in terms of years since original description and the number of peer-reviewed publications, had been the flow cytometric (“FCM”) procedure developed by Nüsse and colleagues (Nüsse et al., “Flow Cytometric Analysis of Micronuclei Found in Cells After Irradiation,” Cytometry 5:20-25 (1984); Schreiber et al., “An Automated Flow Cytometic Micronucleus Assay for Human Lymphocytes,” Int. J. Radiat. Biol. 62:695-709 (1992); Schreiber et al., “Multiparametric Flow Cytometric Analysis of Radiation-Induced Micronuclei in Mammalian Cell Cultures,” Cytometry 13:90-102 (1992); Nüsse et al., “Flow Cytometric Analysis of Micronuclei in Cell Cultures and Human Lymphocytes: Advantages and Disadvantages,” Mutat. Res. 392:109-115 (1997)). The reliability of Nüsse's flow cytometric approach for scoring in vitro MN was improved when adaptations to the two-step detergent method were made by scientists at Litron Laboratories (Avlasevich et al., “In vitro micronucleus scoring by flow cytometry: Differential staining of micronuclei versus apoptotic chromatin enhances assay reliability,” Environ. Mol. Mutagen. 47:56-66 (2006); Bryce et al., 2007, 2008, 2011). The most significant of these advances were incorporation of the fluorescent dye ethidium monoazide bromide (EMA) to differentiate MN from chromatin associated with dead and dying cells, and the use of “counting beads” as a means to derive information about the relative number of healthy cells (a cytotoxicity endpoint) while simultaneously scoring MN.

Further improvements to assay efficiency were realized once procedures were established for conducting cell treatment, staining/lysis, and flow cytometric analysis all in the same 96-well plate. This adaptation requires considerably less compound than conventional in vitro micronucleus test methods, and has a greater compatibility with high throughput screening instrumentation. For instance, Bryce et al. described the use of this approach in conjunction with a robotic auto-sampling device to efficiently test seven genotoxicants over twenty-two closely spaced concentrations (“Miniaturized flow cytometric in vitro micronucleus assay represents an efficient tool for comprehensively characterizing genotoxicity dose-response relationships,” Mutat. Res. 703:191-199 (2010)).

The relative simplicity of the micronucleus endpoint and the availability of automated scoring methods are not the only reasons the in vitro assay is steadily replacing chromosome aberration tests. Another compelling advantage of the micronucleus assay is that it is recognized as having a greater capacity to detect two important types of cytogenetic damage, clastogenicity and aneugenicity (Parry and Parry, “The use of the in vitro micronucleus assay to detect and assess the aneugenic activity of chemicals,” Mutat. Res. 607:5-8 (2006). Conversely, the chromosome aberration assay does not reliably detect aneugens.

While the MN assay is sensitive to both clastogenic and aneugenic agents, in the ordinary conduct of the assay, the MN endpoint alone does not distinguish between these alternate modes of action (MOA). Historically, in order to discriminate between these MOA, there have been requirements to apply additional reagents and processing steps, and this is usually accomplished in a second tier follow-up assay. For instance, the presence of whole chromosomes in MN can be determined via immunochemical labeling of kinetochore proteins (Lynch and Parry, “The cytochalasin-B micronucleus/kinetochore assay in vitro: Studies with 10 suspected aneugens,” Mutat. Res. 287:71-86 (1993)) or fluorescence in situ hybridisation (FISH) using a combination of centromeric and telomeric probes (Doherty et al., “A study of the aneugenic activity of trichlorfon detected by centromere-specific probes in human lymphoblastoid cell lines.” Mutat. Res. 372:221-231 (1996)). This permits chromosome loss and/or non-disjunction (FISH only) to be ascertained and therefore the assay can provide evidence for the underlying MOA. While these techniques provide definitive results, the additional steps and more complex analyses render these approaches less than ideal for routine screening purposes. More efficient methodologies that could simultaneously provide MOA information with MN scoring would be preferred over current approaches.

There have been some reports that for certain cell lines, for instance CHO-K1, flow cytometry-based micronucleus assays may provide genotoxic MOA signatures that are able to discriminate between clastogenic and aneugenic activities (Bryce et al., “Miniaturized flow cytometry-based CHO-K1 micronucleus assay discriminates aneugenic and clastogenic modes of action,” Environ. Mol. Mutagen. 52, 280-286 (2011)). One indicator of aneugenicity that was noted is an increase in the incidence of hypodiploid nuclei. Several mechanisms are expected to contribute to this characteristic—malsegregation of chromosomes via non-disjunction, and the loss of whole chromosome(s) to MN. These activities are predicted to decrease DNA content of daughter nuclei following division, and thus reduce the fluorescence profile to sub-2n status. These nuclei are presumably derived from “healthy cells” due to their lack of EMA staining, a dye that labels cells with compromised membranes prior to the lysis/pan-nucleic acid staining and flow cytometric analysis steps. A secondary marker that may be relatively specific for aneugenicity is an upward shift in the median channel fluorescence of the MN population. This characteristic is believed to arise from the induction of MN derived from whole chromosomes as opposed to fragments generated by clastogens. The paper by Bryce et al. reports that these so-called aneugenic signatures correctly classified 16 genotoxicants as having an aneugenic or clastogenic MOA when the rodent cell line CHO-K1 was used. It is important to note, however, that this same manuscript states that these signatures were not effective for the human lymphoblastoid cell line TK6. A subsequent manuscript by Hashimoto and colleagues suggests that dysfunctional versus functional p53 status may explain some cell lines' lack or presence of a hypodiploid response following exposure to aneugens (Hashimoto et al., “Difference in susceptibility to morphological changes in the nucleus to aneugens between p53-competent and p53-abrogated lymphoblastoid cell lines (TK6 and NH32 cells) in the in vitro micronucleus assay,” Mutagenesis 27:287-293 (2012)). As opposed to hypodiploidy, Hashimoto and colleagues provide evidence that the fraction of cells in metaphase represents a good indicator of aneugenicity in TK6 cells. This conclusion reinforces earlier work that suggested increases in metaphase cells can distinguish between clastogenic and aneugenic MOA, for instance Matsuoka et al., “A proposal for a simple way to distinguish aneugens from clastogens in the in vitro micronucleus test,” Mutagenesis, 14:385-389 (1999) and Muehlbauer et al., “Detection of numerical chromosomal aberrations by flow cytometry: A novel process for identifying aneugenic agents,” Mutat. Res. 585:156-169 (2005). In addition to effects on the frequency of metaphase cells, another recognized indicator of aneugenic activity is the induction of polyploid cells, that is, cells with extra complete set(s) of chromosomes. For example Aardema et al., “Aneuploidy: a report of an ECETOC task force,” Mutat. Res., 410:3-79 (1998), recites that the “majority of definitive aneugens evaluated induce polyploidy in vitro.”

The present invention overcomes the disadvantages of prior art approaches, and satisfies the need of establishing a robust, reliable, high throughput in vitro micronucleus assay that simultaneously acquires data that characterizes treatment-related cytotoxicity, and in the case of MN induction, provides evidence for whether the genotoxic activity is the result of an aneugenic or clastogenic MOA.

### SUMMARY OF THE INVENTION

As used herein, the terms “bundles of metaphase chromosomes”, “metaphase chromosomes”, “metaphase events”, and “H3-positive events” are used interchangeably to describe chromatin in this particular stage of the cell cycle that is characterized by chromatin that has been organized into metaphase chromosomes and lacks a nuclear membrane. These terms are used to differentiate these metaphase events from nuclei associated with all other stages of the cell cycle.

As used herein, the term “nuclei”, as in the case of detergent-liberated nuclei, refers to both nuclear material that is surrounded by a nuclear membrane as well as metaphase chromosomes that lack the nuclear membrane.

As used herein, chromatin associated with dead and/or dying cells is referred to as “chromatin debris”. This is distinguishable from chromatin in nuclei and chromatin in the form of metaphase chromosomes.

A first aspect of the present invention relates to a method for the enumeration of eukaryotic cell micronuclei, while simultaneously acquiring data that is valuable for characterizing cytotoxicity and genotoxicity, and in the case of genotoxicity, for distinguishing between aneugenic and clastogenic modes of action (MOA). This method involves contacting a sample containing eukaryotic cells with a first fluorescent reagent that permeates dead and dying cells but not viable cells, that covalently binds chromatin, and that has a fluorescence emission spectrum; contacting the sample with one or more lysis solutions that result in digestion of eukaryotic cell outer membranes but retention of nuclear membranes, thereby forming free nuclei, micronuclei, bundles of metaphase chromosomes, chromatin debris from dead and/or dying cells, or any combinations thereof; contacting the free nuclei and/or micronuclei and/or metaphase chromosomes and/or chromatin debris with RNase to substantially degrade RNA; contacting the free nuclei and/or micronuclei and/or metaphase chromosomes and/or chromatin debris with a second fluorescent reagent that binds to metaphase chromosome-associated epitopes whose fluorescence emission spectrum does not substantially overlap with the fluorescent emission spectrum of the first fluorescent reagent; staining cellular DNA with a third fluorescent reagent having a fluorescent emission spectrum which does not substantially overlap with the fluorescent emission spectrum of the first or second fluorescent reagents; exciting the first, second, and third fluorescent reagents with light of appropriate excitation wavelength(s); and detecting the fluorescent emission and light scatter produced by the nuclei and/or micronuclei and/or metaphase chromosomes and/or chromatin debris. As a result of such detection, any one or more of the following events can be counted: the number of micronuclei in the sample relative to the number of nuclei, the number of events that exhibit metaphase-specific fluorescence relative to the number of nuclei and/or relative to G2/M (i.e. 4n) nuclei, the number of polyploidy nuclei relative to the number of nuclei, and the number of chromatin debris events relative to the number of nuclei.

A second aspect of the present invention relates to a method for the enumeration of eukaryotic cell micronuclei, while simultaneously acquiring data characterizing cytotoxicity and genotoxicity, and in the case of genotoxicity, for distinguishing between aneugenic and clastogenic modes of action. This method includes: exposing a eukaryotic cell sample comprising whole cells and dead and dying cells to (i) first, second, and third fluorescent reagents that are characterized by fluorescent emission spectra that do not substantially overlap, and (ii) a lysis solution that lyses cellular membranes, said exposing being carried out under conditions effective to allow the first fluorescent reagent to label chromatin debris, the second fluorescent reagent to label metaphase events, and the third fluorescent reagent to label all cellular DNA, including chromatin debris, metaphase chromosomes, nuclei, and micronuclei; exciting the first, second, and third fluorescent reagents; and detecting the fluorescent emission and light scatter produced by the nuclei and/or micronuclei and/or metaphase chromosomes and/or chromatin debris, and counting (i) the number of micronuclei in said sample relative to the number of nuclei, or (ii) the number of metaphase events relative to the number of nuclei and/or G2/M nuclei, or (iii) the number of chromatin debris events relative to the number of nuclei, or (iv) the number of polyploid nuclei relative to the number of nuclei, or any combination thereof, to characterize cytotoxicity and genotoxicity, and in the case of genotoxicity, to distinguish between aneugenic and clastogenic modes of action.

A third aspect of the present invention relates to a method for assessing cytotoxicity or genotoxicity of a chemical or physical agent, and distinguishing between aneugenic and clastogenic modes of action. This method includes: exposing a eukaryotic cell sample, previously exposed to a chemical or physical agent and comprising whole cells and dead and dying cells to (i) first, second, and third fluorescent reagents that are characterized by fluorescent emission spectra that do not substantially overlap, and (ii) a lysis solution that lyses cellular membranes, said exposing being carried out under conditions effective to allow the first fluorescent reagent to label chromatin debris, the second fluorescent reagent to label metaphase events, and the third fluorescent reagent to label all cellular DNA, including chromatin debris, nuclei, metaphase events, and micronuclei, and (iii) a known concentration of counting beads; exciting the first, second, and third fluorescent reagents; and detecting the fluorescent emission and light scatter produced by the nuclei and/or micronuclei and/or chromatin debris and/or metaphase events, and counting beads, and determining one or more of the following endpoints: (i) the frequency of first fluorescent reagent-positive events, usually relative to third fluorescent reagent positive and first fluorescent reagent-negative nuclei, which represents an assessment of cytotoxicity; (ii) the ratio of third fluorescent reagent-positive and first fluorescent reagent-negative nuclei to counting beads, usually represented as a percentage of negative control nuclei to counting bead ratio, which represents an assessment of cytotoxicity; (iii) the frequency of second and third fluorescent reagent-positive and first fluorescent reagent-negative events (i.e., metaphase chromosomes) relative to third fluorescent-positive and first fluorescent reagent-negative nuclei and/or relative to total third fluorescent-positive and first fluorescent reagent-negative G2/M nuclei, whereby decrease(s) relative to a baseline value or negative control represents a measure of cytotoxicity and increase(s) relative to a baseline value or negative control represents an indication of an aneugenic mode of genotoxic activity; (iv) the frequency of third fluorescent reagent-positive and first fluorescent reagent-negative polyploidy nuclei relative to total third fluorescent-positive and first fluorescent reagent-negative nuclei, whereby an increase relative to a baseline value or negative control represents an indication of an aneugenic mode of genotoxic activity; and (v) the proportion of third fluorescent reagent-positive and first fluorescent reagent-negative micronuclei relative to third fluorescent reagent-positive and first fluorescent reagent-negative nuclei as a measure of genotoxicity.

A fourth aspect of the present invention relates to a method of assessing the DNA-damaging potential of a chemical or physical agent. This method involves exposing a sample containing eukaryotic cells to a chemical or physical agent and performing the method according to the first, second, or third aspects of the present invention. A significant increase in the frequency of micronuclei from a baseline micronuclei value in unexposed or negative control eukaryotic cells indicates the genotoxic potential of the chemical or physical agent; a significant decrease in the number of events that exhibit metaphase-specific fluorescence relative to the number of nuclei and/or relative to G2/M nuclei indicates the cytotoxic potential of the chemical or physical agent; a significant elevation in the frequency of metaphase chromosome events relative to the number of nuclei and/or relative to G2/M nuclei from a baseline value in unexposed or negative control eukaryotic cells indicates genotoxicity with an aneugenic MOA; and a significant elevation in the frequency of polyploidy nuclei relative to the number of nuclei from a baseline value in unexposed or negative control eukaryotic cells indicates genotoxicity with an aneugenic MOA.

A fifth aspect of the present invention relates to a method of assessing the cytotoxicity of a chemical or physical agent. This method involves exposing eukaryotic cells to a chemical or physical agent and performing the method according to the first, second, or third aspects of the present invention. A significant increase in the frequency of chromatin debris relative to nuclei from a baseline value in unexposed or negative control eukaryotic cells indicates the cytotoxic potential of the chemical or physical agent, and/or a significant decrease in the number of metaphase events relative to the number of nuclei and/or relative to G2/M nuclei indicates the cytotoxic potential of the chemical or physical agent; and/or a significant decrease in the proportion of nuclei to counting beads, relative to a baseline value in unexposed or negative control eukaryotic cells, indicates the cytotoxic potential of the chemical or physical agent.

A sixth aspect of the present invention relates to a kit that includes: one or more eukaryotic cell membrane lysis solutions; a first fluorescent reagent that permeates dead and dying cells, but not viable cells; a second fluorescent reagent that specifically labels metaphase chromosome-associated epitopes; a third fluorescent reagent that labels all chromatin, where the fluorescent emission spectra of the first, second, and third fluorescent reagents do not substantially overlap; and RNase A solution.

A seventh aspect of the present invention relates to a kit that includes a first lysis solution that comprises NaCl, Na-citrate, and tert-octylphenoxy poly(oxyethylene)ethanol in deionized water; a second lysis solution that comprises citric acid and sucrose in deionized water; ethidium monoazide bromide or propidium monoazide bromide; a fluorescent DNA dye having a fluorescent emission spectrum which does not substantially overlap with a fluorescent emission spectrum of photo-activated ethidium monoazide bromide or propidium monoazide bromide; a fluorochrome-conjugated anti-phosphorylated histone H3 antibody, wherein the fluorochrome has a fluorescent emission spectrum which does not substantially overlap with the fluorescent emission spectrum of photo-activated ethidium monoazide bromide or propidium monoazide bromide, or the fluorescent DNA dye; an RNase A solution; and optionally, one or more of (i) a container comprising an in vitro culture of nucleated eukaryotic cells, (ii) instructions that describe cell harvest and staining procedures, and also scoring via flow cytometry of micronuclei, nuclei, G2/M nuclei, polyploid nuclei, chromatin debris, and metaphase events, (iii) a computer readable storage medium that contains a cytometry data acquisition template for flow cytometric scoring of micronuclei, nuclei, G2/M nuclei, polyploidy nuclei, chromatin debris, and metaphase events; and (iv) counting beads.

The methods described herein provide for the enumeration of eukaryotic cell micronuclei while simultaneously providing information about genotoxic MOA using, preferably, flow cytometry technology. The primary advantage of this methodology relative to other flow cytometry-based procedures is that MN scoring, cytotoxicity, and MOA determinations are made simultaneously, even in cells with normal p53 function, for instance TK6 cells. Thus, the present invention identifies procedures that can be employed for an automated in vitro micronucleus assay that can be used to simultaneously evaluate agents (e.g., chemical or physical agents) for genotoxicity, cytotoxicity, and mode of genotoxic activity to eukaryotic cells. The procedure is fast, reliable, and accurate, and can be performed without the need for dosing of animals. Consequently, significant cost savings can be afforded by the present invention in the process of testing agents for genotoxicity and/or cytotoxicity.

### BRIEF DESCRIPTION OF THE DRAWINGS

FIG. 1 is a schematic illustration of an exemplary cell staining technique according to one embodiment of the present invention. This three fluorescent reagent staining procedure enhances the reliability of flow cytometry-based micronucleus analyses by differentially staining micronuclei and chromatin associated with dead and dying cells, and simultaneously providing information about treatment-related cytotoxicity as well as genotoxic MOA. Ethidium monoazide bromide (“EMA”) represents a preferred first fluorescent reagent, fluorochrome-conjugated anti-phosphorylated histone H3 (e.g., anti-histone H3-pS28 antibody, or anti-H3-P for short) represents a preferred second fluorescent reagent, and SYTOX® Green represents a preferred third fluorescent reagent.

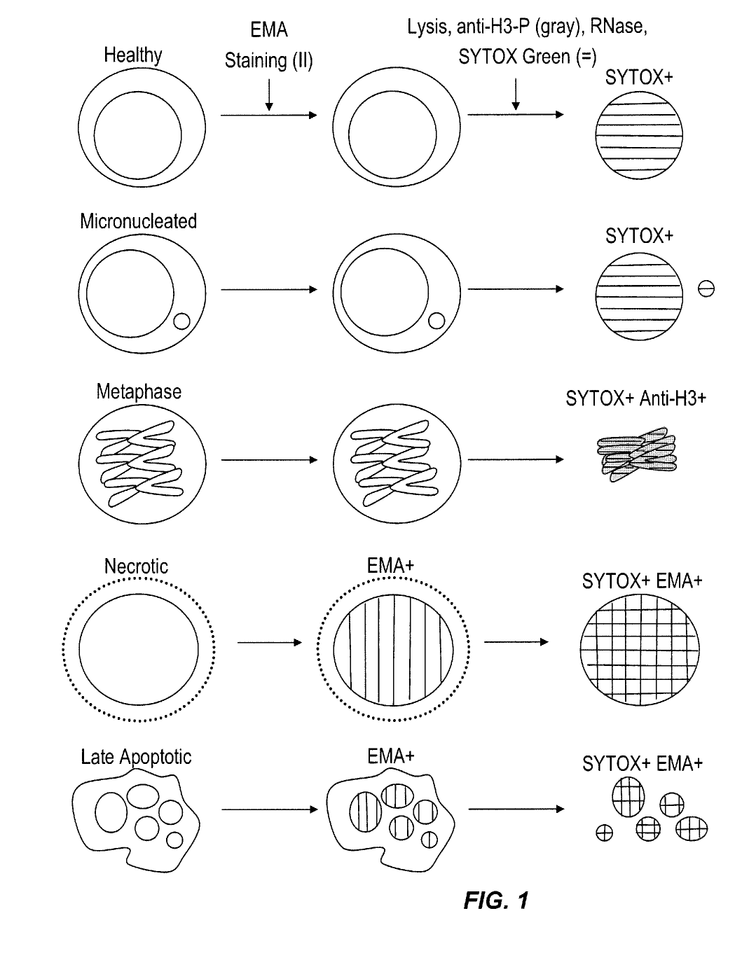

FIG. 2 is a bivariate plot of TK6 cells treated for 5 hrs with 0 or 0.05 μg/ml of the metaphase blocking agent demicolcine (panels A and B, respectively). The cells were then processed according to the present invention whereby nuclei and MN and metaphase chromosomes were liberated with lysis solutions and labeled with SYTOX® Green and anti-H3-P. The liberated nuclei and metaphase chromosomes exhibit SYTOX® Green fluorescence in proportion to their DNA content (X-axis). Even so, this one parameter is not capable of differentiating 4n cells that are in the metaphase versus G2 portion of the cell cycle. Rather, fluorescence associated with anti-H3-P discriminates 4n metaphase cells (region y) from 4n G2 nuclei (Y-axis; region z). The increased percentage of anti-H3-P positive events as a proportion of nuclei (i.e., y/x) and also as a proportion of G2/M cells (i.e., y/(y+z)) in the demicolcine-treated culture provides evidence that the reagent is specifically and effectively labeling chromatin of interest, that is, metaphase chromosomes.

FIGS. 3A-B are graphs showing dose-response relationships for a protypical clastogen (mitomycin C, 3A) and a prototypical aneugen (colchicine, 3B). In this experiment, TK6 cells were treated for 24 continuous hours with a range of mitomycin C or colchicine concentrations, and then cells were processed according to the present invention by exposing cells to the dye EMA, lysing cells and then contacting them with RNase, fluorescent anti-H3-P, and SYTOX® Green. Mean relative survival (RS; derived from nuclei to counting bead ratios, expressed as % of control) and metaphase event frequencies (referred to here as mitotic index, expressed as % of control) are graphed on the Y-axis, and Mean fold increase values for MN and EMA-positive events are graphed on the YY-axis. Mitomycin C is observed to be genotoxic, as increased MN are observed. It is cytotoxic and not aneugenic, as evidenced by dose-dependent reductions to both RS and metaphase events. Colchicine is also observed to be genotoxic, as increased MN are observed. It is cytotoxic, as evidenced by dose-dependent reductions to RS, and its mode of genotoxic action is aneugenicity, which is indicated by an increased proportion of metaphase events.

FIG. 4 depicts the light scatter and fluorescence characteristics of eukaryotic cells processed according to the present invention. For events to be scored as nuclei, MN, or metaphase chromosomes, they were within the illustrated light scatter region, within a SYTOX® Green fluorescence range, and exhibited side light scatter versus SYTOX® Green fluorescence and forward light scatter versus SYTOX® Green fluorescence characteristics of nuclei, MN, or metaphase chromosomes, as shown.

FIG. 5 shows the primary endpoints that are scored in the multiplexed micronucleus assay described herein. The frequency of EMA-positive events represents an assessment of cytotoxicity. The ratio of SYTOX® Green-positive and EMA-negative nuclei to counting beads provides a means to calculate another endpoint of cytotoxicity, relative survival. The frequency of metaphase events, expressed relative to concurrent negative control, or expressed relative to G2/M nuclei, provides information about cytotoxicity and genotoxic mode of action. The proportion of MN relative to SYTOX®Green-positive and EMA-negative nuclei is an endpoint of genotoxicity, and is sensitive to clastogenic and aneugenic activities.

FIG. 6 shows an additional endpoint that is useful for detecting aneugenic activity, that is, the frequency of SYTOX® Green-positive and EMA-negative polyploid nuclei relative to total SYTOX® Green-positive and EMA-negative nuclei. In this example, a substantial treatment-induced increase in the frequency of polyploid nuclei is observed for the aneugen paclitaxel, but not the clastogen cisplatin. As shown here, to successfully enumerate polyploid nuclei, it is desirable to construct data acquisition and analysis templates that include events with at least 8n DNA content.

### DETAILED DESCRIPTION OF THE INVENTION

The present invention is directed to a method for the enumeration of micronuclei in eukaryotic cells using a standard, widely available flow cytometer apparatus which provides for excitation of fluorochromes and detection of resulting fluorescent emissions as well as light scatter signals.

One aspect of the present invention relates to a method for the enumeration of eukaryotic cell micronuclei, while simultaneously acquiring data that is valuable for characterizing cytotoxicity and genotoxicity, and in the case of genotoxicity, for distinguishing between aneugenic and clastogenic modes of action (MOA). This method involves contacting a sample containing eukaryotic cells with a first fluorescent reagent that permeates dead and dying cells but not viable cells, that covalently binds chromatin, and that has a fluorescence emission spectrum; contacting the sample with one or more lysis solutions that result in digestion of eukaryotic cell outer membranes but retention of nuclear membranes, thereby forming free nuclei, micronuclei, bundles of metaphase chromosomes, chromatin debris from dead and/or dying cells, or any combinations thereof; contacting the free nuclei and/or micronuclei and/or metaphase chromosomes and/or chromatin debris with RNase to substantially degrade RNA; contacting the free nuclei and/or micronuclei and/or metaphase chromosomes and/or chromatin debris with a second fluorescent reagent that binds to metaphase chromosome-associated epitopes whose fluorescence emission spectrum does not substantially overlap with the fluorescent emission spectrum of the first fluorescent reagent; staining cellular DNA with a third fluorescent reagent having a fluorescent emission spectrum which does not substantially overlap with the fluorescent emission spectrum of the first or second fluorescent reagents; exciting the first, second, and third fluorescent reagents with light of appropriate excitation wavelength(s); and detecting the fluorescent emission and light scatter produced by the nuclei and/or micronuclei and/or metaphase chromosomes and/or chromatin debris. As a result of such detection, any one or more of the following events can be counted: the number of third fluorescent reagent-positive and first fluorescent reagent-negative micronuclei in the sample relative to the number of third fluorescent reagent-positive and first fluorescent reagent-negative nuclei; the number of first and third fluorescent reagent-positive events (i.e., chromatin debris) is counted, optionally relative to the number of third fluorescent reagent-positive and first fluorescent reagent-negative nuclei; the number of second and third fluorescent reagent-positive and first fluorescent reagent-negative events (i.e., metaphase events) relative to the number of third fluorescent reagent-positive and first fluorescent reagent-negative nuclei and/or relative to G2/M (i.e. 4n) nuclei; and/or the number of third fluorescent reagent-positive and first fluorescent reagent-negative polyploid nuclei relative to the number of third fluorescent reagent-positive and first fluorescent reagent-negative nuclei.

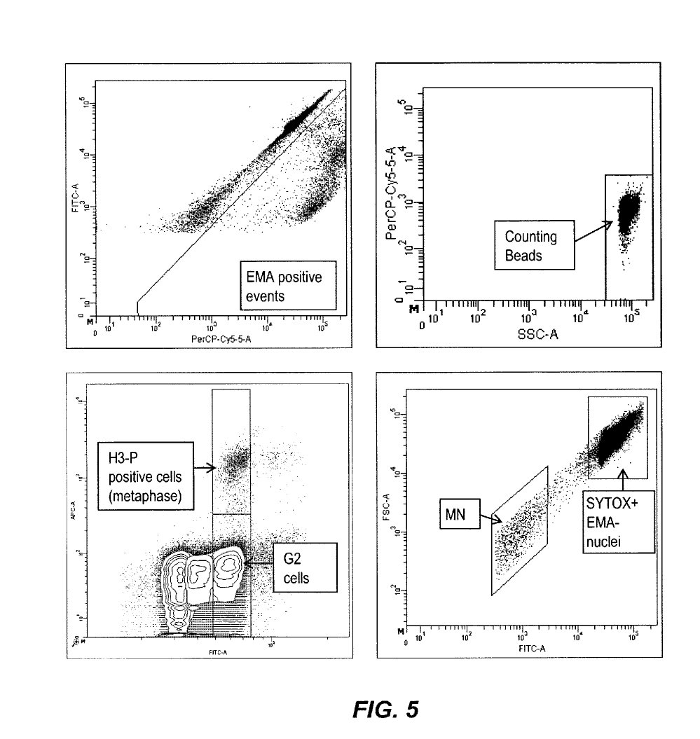

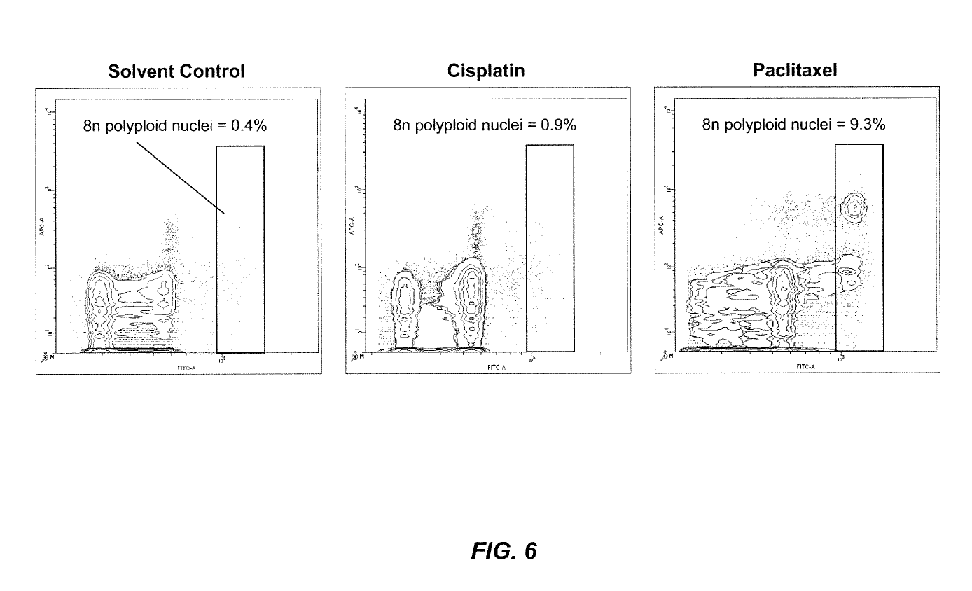

As noted in the accompanying Examples (see FIGS. 5 and 6), a number of different endpoints can be assessed. The frequency of third and first fluorescent reagent-positive events represents an assessment of cytotoxicity. Through the use of a known concentration of counting beads, the ratio of counting beads to third fluorescent reagent-positive and first fluorescent reagent-negative nuclei provides a means to calculate another endpoint of cytotoxicity, relative survival. The frequency of third and first fluorescent reagent-positive events (i.e., chromatin debris) represents an assessment of cytotoxicity. The frequency of metaphase events provides information about cytotoxicity and genotoxic mode of action. The frequency of polyploid nuclei is responsive to aneugenic activity. The proportion of MN relative to third fluorescent reagent-positive and first fluorescent reagent-negative nuclei is an endpoint of genotoxicity, and is sensitive to clastogenic and aneugenic activities.

As indicated above, the frequency of micronuclei, nuclei, metaphase events, polyploid nuclei, and chromatin debris can be expressed relative to other populations, for instance metaphase chromosomes can be expressed as a percentage of first fluorescent reagent-negative and third fluorescent reagent-positive nuclei or first fluorescent reagent-negative and third fluorescent reagent-positive G2/M nuclei. Alternatively, micronuclei, nuclei, metaphase events, polyploidy nuclei, and chromatin debris can be expressed per unit volume of sample or per unit time (based on the fluidic rate and the time taken to analyze the sample). Alternatively, counting beads can be added to the sample and the fluorescent emission and light scatter of the counting beads is detected and enumerated along with the other events to obtain an event-to-bead ratio. The counting beads can be a suspension of latex particles or similar uniform particle that can be readily differentiated from the cells. Preferred latex particles include, without limitation, CountBright™ Absolute Counting Beads and 6 micron Peak Flow™ fluorescent microspheres from Life Technologies. In one embodiment of the present invention, such counting beads are added after use of the lysis solutions and labeling with the fluorescent reagents. However, it will be appreciated by those knowledgeable in the art that there are alternate and equally acceptable times during the labeling procedure when counting beads can be added and used effectively to obtain the desired relative survival or other values.

Eukaryotic cells suitable for carrying out the methods of the present invention include any types of animal cells, preferably mammalian cells, as well as plant protoplasts. Exemplary animal cells suitable for carrying out the methods of the present invention include, without limitation, immortalized cell lines, as well as cells which have only recently been harvested from animal species (e.g., primary cell cultures).

Preferred primary cell cultures are those that divide in culture (i.e., with appropriate growth media, which for some cell types requires the inclusion of cytokines and/or other factors such as mitogens). Exemplary cell types that can be screened easily using the methods of the present invention include, without limitation, blood-, spleen-, lymph node-, or thymus-derived lymphocytes, bone marrow-derived stem cells, and hepatocytes.

Exemplary immortalized cell lines, include, without limitation, L5178Y, AHH-1, WIL-2NS, HepG2, HepRG, MCL-5, CHO-K1, and TK6 cells (Zhan et al., Genotoxicity of Microcystin-LR in Human Lymphoblastoid TK6 Cells,” Mutat. Res. 557:1-6 (2004), which is hereby incorporated by reference in its entirety). Each of these cell types has been used in genotoxicity investigations.

Micronuclei are membrane-bound, extra-nuclear, sub-2n DNA structures resulting from double-strand chromosome breaks or from the dysfunction of mitotic spindle apparatus. Micronuclei are also known as Howell-Jolly bodies in the hematology literature.

Chromatin of dead and/or dying cells is DNA derived from cells which are no longer viable, or from cells which have progressed to an irreversible stage of cell death. Thus, “dead and/or dying cells” and “chromatin debris” is meant to encompass necrotic cell death typified by cytoplasmic swelling and rupture, as well as apoptotic cell death which is usually characterized by cellular and nuclear shrinkage, condensation of chromatin, and fragmentation of nuclei.

The first fluorescent reagent can be any dye that can permeate the dead and/or dying cells but not viable cells, and covalently bind chromatin. Preferably, the first fluorescent reagent is, at the time of contacting the cells in culture, in an inactive form. Thereafter, the reagent is activated to a reactive form, which is controlled by conditions that can be easily manipulated in a laboratory setting (e.g., by light activation, change in pH, etc.). Upon activation, the reagent should bind covalently to DNA, i.e., chromatin. When the reagent is covalently bound to the DNA of dead and/or dying cells, it changes the nature of staining away from an equilibrium situation. In particular, this approach for staining ensures that the fluorescent signal that is imparted to dead and/or dying cells is not diminished during subsequent cell processing steps. In a preferred embodiment, the first fluorescent reagent is the DNA dye ethidium monoazide bromide (“EMA”), which is efficiently converted to a reactive form through photoactivation. Propodium monoazide bromide (“PMA”) is another suitable DNA dye that is efficiently converted to a reactive form through photoactivation.

The one or more lysis solutions can be any suitable lysis solution, or combination thereof, for cell membrane lysis. According to one embodiment, first and second lysis solutions are provided, with the first lysis solution having NaCl, Na-citrate, and octylphenyl-polyethylene glycol (IGEPAL®, Sigma) in deionized water and the second lysis solution having citric acid and sucrose in deionized water. Cell lysis preferably occurs according to modifications to a procedure that has been described in the literature (Nüsse et al., “Flow Cytometric Analysis of Micronuclei Found in Cells After Irradiation,” Cytometry 5:20-25 (1984); Nüsse et al., “Factors Influencing the DNA Content of Radiation-Induced Micronuclei,” Int. J. Radiat. Biol. 62:587-602 (1992); and Nüsse et al., “Flow Cytometric Analysis of Micronuclei in Cell Cultures and Human Lymphocytes: Advantages and Disadvantages,” Mutat. Res. 392:109-115 (1997), which are hereby incorporated by reference in their entirety). In particular, these two solutions are preferably used sequentially, as described in the accompanying Examples.

In one embodiment of the methods of the present invention, contacting the sample with one or more lysis solutions and contacting the free nuclei and/or MN and/or metaphase chromosomes and/or chromatin debris with RNase may be carried out simultaneously. Alternatively, these steps are carried out sequentially.

Suitable second fluorescent reagents specifically bind to metaphase chromosome-specific epitopes, and have a fluorescent emission spectrum that does not significantly overlap with the emission spectrums of the first and third fluorescent reagents. A preferred second fluorescent reagent is fluorochrome-conjugated antibody that binds specifically to phosphorylated histone H3. Several antibodies to phosphorylated histone H3 are suitable for this purpose, including those that bind to H3 phosphorylated at serine 139, serine 28 and/or serine 10 (referred to herein as anti-H3-P). In this case, the epitope that is recognized is a phosphorylated form of histone H3. Since H3 phosphorylation of serines 10, 28, and 139 is tightly controlled and strictly associated with the metaphase portion of the cell cycle, anti-H3-P represents a specific signal for bundles of metaphase chromosomes of the type liberated by the lysis procedures described herein. Any other antibody that is specific for metaphase chromosomes can be utilized.

In a preferred embodiment of the methods of the present invention, contacting the sample with anti-H3-P occurs in conjunction with a detergent-containing lysis solution that facilitates proper recognition and binding of the anti-H3-P fluorescent reagent to the H3-P epitope. This antibody-epitope interaction tends to require pH in a range of approximately 6 to 8, and hence said contacting is preferably performed in conjunction with the first lysis solution of the type described above and not in conjunction with a second lysis solution described above, which has a lower pH.

In one embodiment of the present invention, contacting the sample with one or more lysis solutions, contacting the free nuclei and/or MN and/or metaphase chromosomes and/or chromatin debris with RNase, contacting the free nuclei and/or MN and/or metaphase chromosomes and/or chromatin debris with a fluorescent reagent that binds metaphase chromosome-specific epitopes, and staining cellular DNA with a third fluorescent DNA dye are carried out simultaneously. Alternatively, these steps are carried out sequentially.

Suitable third fluorescent reagents are capable of staining cellular DNA at a concentration range detectable by flow cytometry, and have a fluorescent emission spectrum that does not substantially overlap with the fluorescent emission spectrum of the first or second fluorescent reagents. It should be appreciated by those of ordinary skill in the art that other nucleic acid dyes are known and are continually being identified. Any suitable nucleic acid dye with appropriate excitation and emission spectra can be employed, preferably cyanine dyes such as YO-PRO®-1, SYTOX® Green, SYBR® Green I, SYTO®11, SYTO®12, SYTO®13, SYTO®59, BOBO®, YOYO®, and TOTO®. Preferred third fluorescent reagents are pan-DNA dyes, one of which is SYTOX® Green.

The first, second, and third fluorescent reagents have sufficiently distinct emission maxima. Preferably, at least two of these fluorescent reagents have similar excitation spectra. The advantage of shared excitation spectra is that it affords the use of widespread dual-laser flow cytometers to execute this three fluorochrome method. For example, when the first fluorescent reagent is EMA and the third fluorescent reagent is SYTOX® Green, both the first and third fluorescent reagents are sufficiently excited by a flow cytometer equipped with a 488 nm laser. Then, for example, when the second fluorescent reagent is conjugated to allophycocyanin (APC), a common second (red diode) laser provides sufficient excitation of the metaphase chromosome-specific reagent. Moreover, it is preferable that the first, second, and third fluorescent reagents do not exhibit fluorescent resonance energy transfer that may interfere with detection emissions by any one of these reagents.

Single-laser flow cytometric analysis uses a single focused laser beam with an appropriate emission band to excite the first and second fluorescent DNA dyes. As stained nuclei, micronuclei, metaphase-chromosomes, and chromatin debris pass through the focused laser beam, they exhibit a fluorescent emission maxima characteristic of the fluorescent dye(s) associated therewith. Dual- or multiple-laser flow cytometric analysis uses two or more focused laser beams with appropriate emission bands in much the same manner as described for the single-laser flow cytometer. Different emission bands afforded by the two or more lasers allow for additional combinations of fluorescent reagents to be employed, and represents a preferred analytical platform for conducting the multiplexed analyses described herein.

Preferably, the flow cytometer is equipped with appropriate detection devices to enable detection of the fluorescent emissions and light scatter produced by the nuclei, MN, metaphase chromosomes, and chromatin debris. These “light scatter” signals serve as additional criteria which helps discriminate nuclei, micronuclei, metaphase chromosomes, chromatin debris, and other subcellular debris from one another. The use of light scatter parameters to serve as additional criteria for accurately measuring micronuclei by flow cytometry has been described in the literature (Nüsse et al., “Flow Cytometric Analysis of Micronuclei in Cell Cultures and Human Lymphocytes: Advantages and Disadvantages,” Mutat. Res. 392:109-115 (1997), which is hereby incorporated by reference in its entirety).

A further aspect of the present invention relates to a method of assessing the DNA-damaging potential of a chemical or physical agent. This method involves exposing a sample containing eukaryotic cells to a chemical or physical agent and performing a method for the enumeration of eukaryotic cell micronuclei of the present invention. A significant increase in the frequency of MN from a baseline MN value in unexposed or negative control eukaryotic cells indicates the genotoxic potential of the chemical or physical agent, a significant decrease in the number of metaphase events relative to the number of nuclei and/or 4n nuclei indicates the cytotoxic potential of the chemical or physical agent, a significant increase in the number of events that exhibit metaphase events relative to the number of nuclei and/or 4n nuclei provides evidence for an aneugenic mode of action of the chemical or physical agent, and a significant increase in the number of polyploid nuclei relative to nuclei provides evidence for an aneugenic mode of action of the chemical or physical agent.

Physical agents which are known to damage DNA include, without limitation, ionizing radiation, such as gamma and beta radiation, and UV radiation.

Chemical agents which are known to damage DNA include, without limitation, inorganic genotoxicants (e.g., arsenic, cadmium and nickel), organic genotoxicants (especially those used as antineoplastic drugs, such as cyclophosphamide, cisplatin, vinblastine, cytosine arabinoside, and others), anti-metabolites (especially those used as antineoplastic drugs, such as methotrexate and 5-fluorouracil), organic genotoxicants that are generated by combustion processes (e.g., polycyclic aromatic hydrocarbons such as benzo(a)pyrene), certain protein kinase inhibitors, as well as organic genotoxicants that are found in nature (e.g., aflatoxins such as aflatoxin B1).

The methods of the present invention are suitable for assessing the DNA-damaging potential of both physical and chemical agents, either alone or in combination with other such agents. For example, physical and chemical agents that are under current investigation for therapeutic treatment, or agents that are being screened for potential therapeutic treatment are amenable to the methods of the present invention.

In carrying out the methods of the present invention, exposure of eukaryotic cells to physical or chemical agents is preferably carried out for a predetermined period of exposure time. Suitable exposure time for detecting chromosome breaking (i.e., clastogenic) agents is between about 3 and about 24 hours, although more or less time may be suitable for some agents. There are some reports which suggest that a preferred exposure time for detecting aneugenic agents is approximately 24 hours (Phelps et al., “A Protocol for the In Vitro Micronucleus Test. II. Contributions to the Validation of a Protocol Suitable for Regulatory Submissions from an Examination of 10 Chemicals with Different Mechanisms of Action and Different Levels of Activity,” Mutat. Res. 521:103-112 (2002), which is hereby incorporated by reference in its entirety), although more or less time may be suitable for some agents.

Methods of assessing the DNA-damaging potential of a physical or chemical agent may further involve a delay between the end of exposure and prior to performing cell harvest, contacting or staining with the several fluorescent reagents, membrane lysis, and flow cytometric analysis according to the previously described methods of the present invention. When employed, the delay or “recovery” period is preferably between about 5 minutes to about 24 hours, although longer or shorter delays can also be utilized.

To some degree, exposure time and recovery periods will be cell line- and chemical class-dependent. Persons of skill in the art can readily optimize the methods of the present invention for different types of eukaryotic cells and different physical or chemical agents.

Certain agents may offer protection from DNA damage, while others magnify risk of damage. The present invention can be used to evaluate the effects of an agent which can modify (i.e., enhance or suppress) such damage. To assess the suspected protective effects of an agent, it can be added to the culture of cells prior to, concurrently with, or soon after addition of a known genotoxicant. Any protective effect afforded by the agent can be measured relative to damage caused by the genotoxicant agent alone. For example, putative protective agents can be vitamins, bioflavonoids and anti-oxidants, dietary supplements (e.g., herbal supplements), or any other protective agent, naturally occurring or synthesized by man.

To assess the ability of an agent to synergistically or additively enhance genotoxicity, the agent can be added to the culture of cells prior to, concurrently with, or shortly after addition of a known genotoxicant. Any additive or synergistic effect caused by the agent can be measured relative to damage caused by the genotoxicant agent alone.

Concurrent cytotoxicity assessment of chemical and/or physical agents (with or without protective agents or enhancing agents) can also be made, pursuant to the methods of the present invention, such as (i) cell cycle effects based on the fluorescence intensity of the third fluorescent reagent which is exhibited by first fluorescent reagent-negative nuclei, (ii) cell cycle effects based on the fluorescence intensity of the second and third fluorescent reagents such that the percentage of metaphase cells can be determined, and (iii) cytotoxicity based on the percentage of particles that exhibit fluorescence associated with the first fluorescent reagent.

Thus, another aspect of the present invention relates to a method of assessing the cytotoxicity of a chemical or physical agent. This method involves exposing eukaryotic cells to a chemical or physical agent and performing the method for the enumeration of eukaryotic cell micronuclei of the present invention. A significant deviation to the normal cell cycle, and/or a significant increase in the frequency of chromatin debris and/or a significant decrease in the proportion of metaphase events from a baseline value in unexposed or negative control eukaryotic cells indicates the cytotoxic potential of the chemical or physical agent. In one embodiment, cytotoxicity can be assessed by measuring relative cell survival. In accordance with this embodiment, counting beads can be used as described in the accompanying Examples to obtain an accurate assessment of relative cell survival following cell exposure to a chemical of physical agent.

Another aspect of the present invention relates to a method of evaluating the effects of an agent which can modify endogenously-induced DNA damage. This method of the present invention can be carried out by exposing eukaryotic cells to an agent that may modify endogenously-induced genetic damage to eukaryotic cells. The method for the enumeration of eukaryotic cell micronuclei of the invention is then performed with the exposed eukaryotic cells. A significant deviation in the frequency of MN from a baseline MN value in unexposed or negative control cells indicates that the agent can modify endogenous DNA damage, a significant change in the number of metaphase events relative to the number of nuclei and/or 4n nuclei indicates that the agent can modify endogenously-induced cytotoxicity and/or aneugenicity, and a significant change in relative survival compared to unexposed or negative control cells indicates that the agent can modify endogenous cytotoxicity.

A further aspect of the present invention relates to a method of evaluating the effects of an agent which can modify exogenously-induced DNA damage. This method of the present invention can be carried out by exposing eukaryotic cells to an exogenous agent that causes genetic damage and an agent that may modify exogenously-induced genetic damage. The method for the enumeration of eukaryotic cell micronuclei of the present invention is then performed with the exposed eukaryotic cells. A significant deviation in the frequency of micronuclei from genotoxicant-exposed eukaryotic cells indicates that the agent can modify exogenously-induced DNA damage, a significant change in the number of metaphase events compared to genotoxicant-exposed eukaryotic cells indicates that the agent can modify exogenously-induced cytotoxicity and/or aneugenicity, and a significant change in relative survival compared to genotoxicant-exposed eukaryotic cells indicates that the agent can modify exogenously-induced cytotoxicity.

Yet another aspect of the present invention relates to a kit that includes: one or more eukaryotic cell membrane lysis solutions; first, second, and third fluorescent reagents as described above; and RNase A solution.

The kit may also include instructions that describe cell harvest, cell staining or contacting procedures, and micronucleus scoring via flow cytometry. The kit may also include a computer readable storage medium that contains a cytometry data acquisition template for flow cytometric micronucleus scoring, including the scoring of all of the events described above as well as the relative frequencies of one type of event to another. The kit may also contain counting beads, typically in the form of a solution or mixture. A container having an in vitro culture of eukaryotic cells may also be included in the kit of the present invention.

### EXAMPLES

The examples below are intended to exemplify the practice of the present invention but are by no means intended to limit the scope thereof.

### Materials and Methods

Chemicals, Fluorescent Reagents, and Miscellaneous Supplies

The identities of the eighteen chemicals evaluated in the following examples, as well as solvent and other information, are listed in Table 1.

TABLE I
List of Reference Genotoxicants That Were Studied

| | | | Genotoxic |
|---|---|---|---|
| Chemical (Abbreviation) | Cas No. | Solvent | Mode of Action |
| Methyl methanesulfonate | 66-27-3 | DMSO | Clastogen |
| (MMS) | | | |
| Hydroxyurea (HU) | 127-07-1 | DMSO | Clastogen |
| Etoposide | 33419-42-0 | DMSO | Clastogen |
| Mitomycin C (MMC) | 50-07-7 | Media | Clastogen |
| Cytosine Arabinoside (Ara-C) | 147-94-4 | DMSO | Clastogen |
| 5 Flurouracil (5-FU) | 51-21-8 | DMSO | Clastogen |
| Bleomycin | 9041-93-4 | DMSO | Clastogen |
| Camptothecin | 7689-03-4 | DMSO | Clastogen |
| Aphidicolin | 38966-21-1 | DMSO | Clastogen |
| N-Methyl-N′-nitro-N- | 70-25-7 | DMSO | Clastogen |
| nitrosoguanidine (MNNG) | | | |
| Cisplatin | 15663-27-1 | DMSO | Clastogen |
| Vinblastine sulfate | 143-67-9 | DMSO | Aneugen |
| Carbendazim | 10605-21-7 | DMSO | Aneugen |
| Colchicine | 64-86-8 | DMSO | Aneugen |
| Vincristine sulfate | 2068-78-2 | DMSO | Aneugen |
| Paclitaxel | 33069-62-4 | DMSO | Aneugen |
| Griseofulvin | 126-07-8 | DMSO | Aneugen |
| Dithylstilbestrol (DES) | 56-53-1 | DMSO | Aneugen |
| Demecolcine | 477-30-5 | DMSO | Aneugen |

DMSO = dimethyl sulfoxide

Each of these eighteen chemicals was purchased from Sigma-Aldrich Corp. (St. Louis, Mo.). DMSO (CAS No. 67-68-5), IGEPAL® CA-630 (CAS No. 9036-19-5), propidium iodide (CAS No. 25535-16-4), sodium citrate (CAS No. 6132-04-3), citric acid (CAS No. 77-92-9), and sucrose (CAS No. 57-50-1), were also obtained from Sigma-Aldrich Corp. RNase A was from Biomatik Corp. NaCl (CAS No. 7647-14-5) was purchased from J. T. Baker (Phillipsburg, N.J.). The fluorescent dyes ethidium monoazide bromide (cat. no. E1374) and SYTOX® Green (cat. no. 57020) were from AnaSpec, Femont, Calif. and Life Technologies, Carlsbad, Calif., respectively. Anti-H3-P antibody was purchased from BD Biosciences, San Jose, Calif. Phosphate buffered saline (PBS) and heat-inactivated fetal bovine serum were from MediaTech Inc., Manassas, Va. Peak Flow™ fluorescent microspheres (6 micron; cat. no. P14828) were purchased from Life Technologies, and served as counting beads as described below.

Cells and Culture Medium

The TK6 cells used in these studies were from American Type Tissue Collection (ATCC) (Manassas, Va.). Cells were maintained in culture medium at 37° C., 5% CO₂, and in a humid atmosphere. Cells were maintained between approximately 1×10⁴ and 1×10⁶ cells/ml for routine passage. The culture medium consisted of RPMI 1640 supplemented with 2 mM L-glutamine, 100 IU penicillin and 100 μg/ml streptomycin, to which heat inactivated horse serum was added for 10% v/v final concentration (all from MediaTech Inc., Herndon, Va.).

Treatment with Demecolcine and Assessment of Anti-H3-P Performance

TK6 cells were treated with 0 or 0.05 ng demecolcine/ml in T25 tissue culture flasks. At the start of treatment, cells were at 5×10⁵/ml in a volume of 20 ml per flask. Flasks were incubated at 37° C., 5% CO₂, and in a humid atmosphere. After 5 hours, flasks were removed and 1 ml aliquots were placed in to 15 ml centrifuge tubes. Cells were collected via centrifugation, and supernatants aspirated such that approximately 25 μl of supernatant remained per tube. Cells were gently resuspended with tapping. 300 μl “Lysis Solution 1” was added slowly to each tube (approximately 5 seconds per sample). Lysis Solution 1 was prepared with deionized water and 0.584 mg/ml NaCl, 1 mg/ml sodium citrate, 0.3 μl/ml IGEPAL® (Sigma), 1 mg/ml RNase A, 0.4 μM SYTOX® Green, and 0.3125-5 μl/ml anti-H3-P (conjugated to Alexa 647). Upon addition of Lysis Solution 1, the tubes were briefly vortexed. These samples were kept at room temperature. At 1 hr, 300 μl “Lysis Solution 2” was injected forcefully into each tube, which were immediately vortexed for 5 seconds. Lysis Solution 2 was prepared with deionized water and 85.6 mg/ml sucrose, 15 mg/ml citric acid, and 0.4 μM SYTOX®Green. These specimens were maintained at room temperature for 30 minutes. Subsequently, samples were stored at room temperature until flow cytometric analysis (same day).

Treatment with Each of 17 Reference Genotoxicants

TK6 cells were treated over a range of chemical concentrations in 24 well plates. At the start of treatment, cells were at 2×10⁵/ml in a volume of 1 ml per well. Before being added to wells, counting beads were added to the culture at a concentration of approximately 1 drop per 10 ml. These fluorescent particles provided a means to calculate relative survival values (relative to solvent control) at the time of harvest. Continuous treatment occurred for 24 to 27 hrs, during which time plates were incubated at 37° C., 5% CO₂, and in a humid atmosphere. Each concentration of chemical was studied in three replicate wells.

Processing of Genotoxicant-Exposed Cultures

At the time of cell harvest, cells were resuspended and 1 ml aliquots were transferred to deep well U bottom 96 well plates. Cells were collected via centrifugation at approximately 340×g for 5 minutes. Supernatants were aspirated, and cells were resuspended with gentle tapping of the plate. Plates were placed on wet ice for 20 minutes with 300 μl EMA dye solution per well. EMA dye solution was composed of PBS with 2% v/v heat-inactivated fetal bovine serum and EMA at 8.75 μg/ml. The plates were submerged to a depth of approximately 2 cm in crushed ice. A fluorescent light source was positioned approximately 30 cm above the plates for 30 minutes.

After the photoactivation period, 700 μl cold PBS with 2% v/v heat-inactivated fetal bovine serum was added to each sample. Cells were collected via centrifugation, and supernatants aspirated such that approximately 25 μl of supernatant remained per well. Cells were gently resuspended with tapping. 300 μl Lysis Solution 1 was added slowly to each well (approximately 3 seconds per sample). Lysis Solution 1 was prepared with deionized water and 0.584 mg/ml NaCl, 1 mg/ml sodium citrate, 0.3 μl/ml IGEPAL® (Sigma), 1 mg/ml RNase A, 0.4 μM SYTOX® Green, and 0.3125-1 μl/ml anti-H3-P (Alexa 647 conjugate). Upon addition of Lysis Solution 1, the wells were pipetted up and down to mix samples, after which time they were maintained at room temperature. After 1 hr, 300 μl “Lysis Solution 2” was injected forcefully into each well, and this volume was immediately pipetted up and down several times. Lysis Solution 2 was prepared with deionized water and 85.6 mg/ml sucrose, 15 mg/ml citric acid, and 0.4 μM SYTOX® Green. These specimens were maintained at room temperature for up to 24 hrs until flow cytometric analysis.

Flow Cytometric Analyses

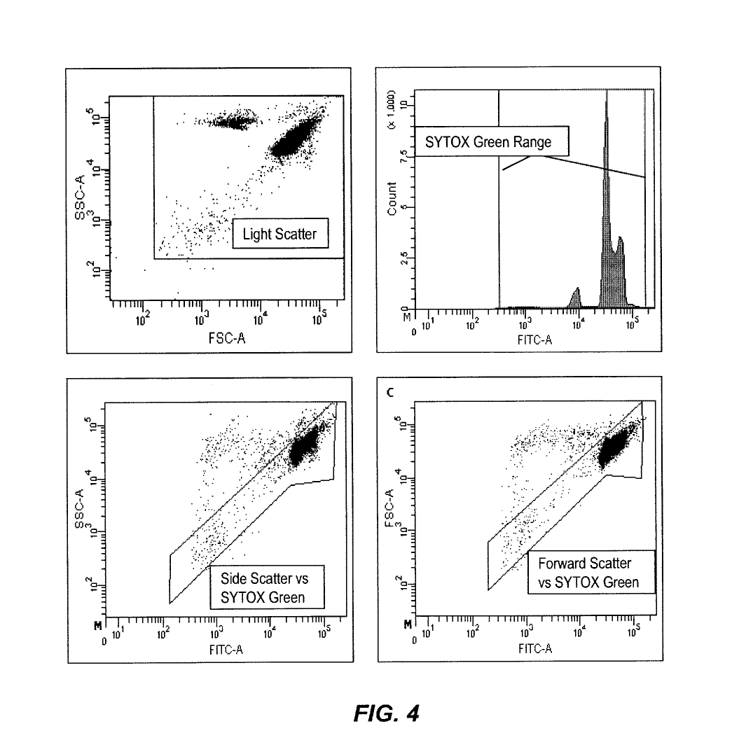

Samples were gently pipetted to resuspend the particles. Data acquisition and analysis was then accomplished with a dual-laser flow cytometer, 488 nm and 633 nm excitation (FACSCantoII, BD Biosciences, San Jose, Calif.). Instrumentation settings and data acquisition/analysis were controlled with Diva software v6.1.3. SYTOX®-associated fluorescence emission was collected in the FITC channel, EMA-associated fluorescence was collected in the PerCP-Cy5 channel, and anti-H3-P-Alexa 647 associated fluorescence was collected in the APC channel. Events were triggered on FITC fluorescence. The flow cytometry gating strategy that was developed for this scoring application required events to meet each of several separate criteria before they were scored as nuclei, MN, chromatin debris, or metaphase chromosomes. See FIGS. 4 and 5. The incidence of flow cytometric-scored MN is expressed as frequency percent (number of SYTOX® Green-positive and EMA-negative MN events/number SYTOX® Green-positive and EMA-negative nuclei×100); relative survival was based on ratios of SYTOX® Green-positive and EMA-negative nuclei to counting beads, and is relative to the mean ratio observed in the negative control wells; the incidence of EMA-positive events is expressed as frequency percent (number of EMA-positive events/number of SYTOX® Green-positive events×100); the incidence of metaphase events is expressed as frequency percent (number of metaphase events/number of SYTOX® Green-positive and EMA-negative nuclei×100); the incidence of metaphase events is also expressed relative to the percentage of G2/M (number of metaphase events/number of G2/M events×100). These calculations were based on the acquisition times of approximately 4 minutes, which was generally sufficient to collect 2,000 or more anti-H3-P-Alexa 647-positive events per replicate.

Polyploidy as an Aneugenic Signature

In this experiment, TK6 cells were treated with solvent (DMSO) or 3.125 μg cisplatin/ml or 0.025 μg paclitaxel/ml. Treatments occurred in wells of a 96-well plate, demonstrating the ability to scale the invention to smaller vessels and thereby reduce the amount of test article required. At the start of treatment, cells were at 2×10⁵/ml in a volume of 300 μl per well. Continuous treatment occurred for approximately 24 hrs, during which time plates were incubated at 37° C., 5% CO₂, and in a humid atmosphere. Each treatment condition was studied in three replicate wells.

After 24 hrs of incubation, cells were collected via centrifugation at approximately 340×g for 5 minutes. Supernatants were aspirated, and cell pellets were resuspended with gentle tapping of the plate. Plates were placed on wet ice for 20 minutes with 50 μl EMA dye solution per well. EMA dye solution was composed of PBS with 2% v/v heat-inactivated fetal bovine serum and EMA at 8.75 μg/ml. The plates were submerged to a depth of approximately 2 cm in crushed ice. A fluorescent light source was positioned approximately 30 cm above the plates for 30 minutes.

After the photoactivation period, 150 μl cold PBS with 2% v/v heat-inactivated fetal bovine serum was added to each well. Cells were collected via centrifugation, and supernatants aspirated. Cell pellets were gently resuspended with tapping. 75 μl Lysis Solution 1 was added to each well (deionized water and 0.584 mg/ml NaCl, 1 mg/ml sodium citrate, 0.3 μl/ml IGEPAL®, 1 mg/ml RNase A, 0.4 μM SYTOX® Green, and 0.3125-1 μl/ml anti-H3-P-Alexa 647). Upon addition of Lysis Solution 1, the wells were pipetted up and down to mix samples, after which time they were maintained at room temperature. After 1 hr, 75 μl Lysis Solution 2 was injected into each well (deionized water and 85.6 mg/ml sucrose, 15 mg/ml citric acid, and 0.4 μM SYTOX® Green). These specimens were maintained at room temperature until flow cytometric analysis occurred (within 24 hrs).

Flow cytometric analysis occurred as described above for the 18 reference test articles with one exception. Rather than acquiring data for 4 minutes, the stop mode was set for 20,000 nuclei.

### Example 1

### Treatment with Demecolcine

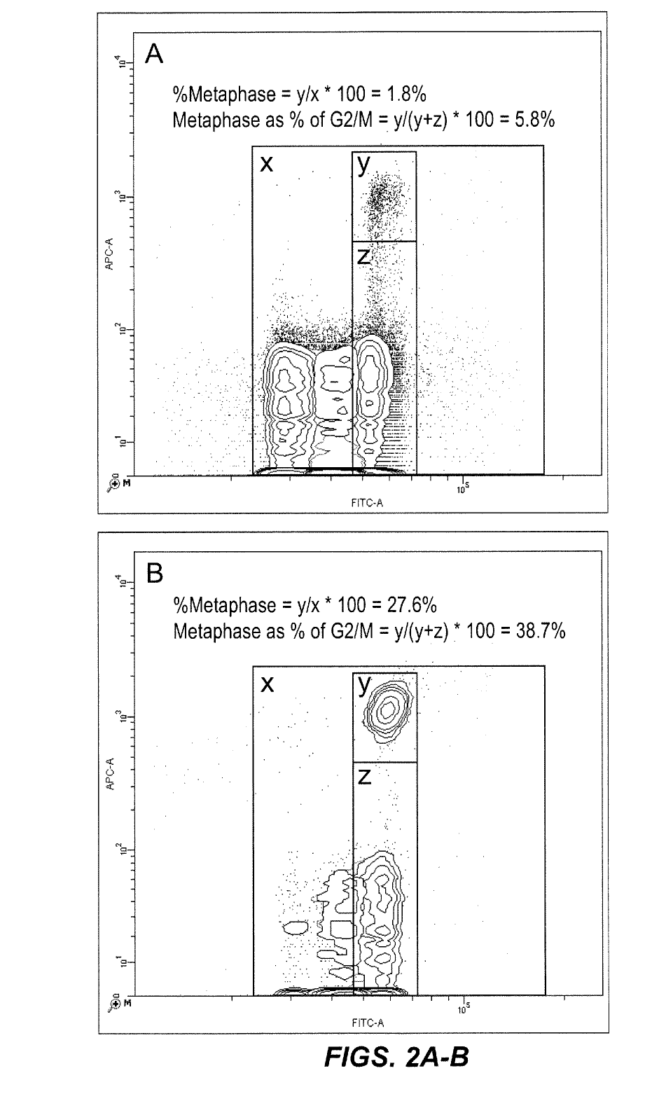

In this experiment, TK6 cells were treated with 0 or 0.05 ng/ml demecolcine, a metaphase blocking agent. As described above, the cells were then contacted with a first lysis solution that brought them simultaneously in contact with detergent, SYTOX® Green as a pan-DNA fluorescent dye, anti-H3-P (Alexa 647 conjugate) as a metaphase specific fluorochrome, and with RNase. After an appropriate incubation period, the liberated nuclei, MN, metaphase chromosomes, and chromatin debris were contacted with a second lysis solution, and then analyzed on a dual-laser flow cytometer. FIG. 2 shows the resulting bivariate plots of anti-H3-P versus SYTOX® Green fluorescence. Whereas the SYTOX® Green parameter alone is able to differentiate G1, S, and G2/M cells based on their increasing nucleic acid dye associated fluorescence, it is not capable of differentiating metaphase events from G2/M, as both exhibit 4n DNA content. However, the anti-H3-P associated fluorescence signal is able to differentiate between G2 and metaphase chromosomes. The fact that demecolcine treatment greatly increased the frequency of 4n anti-H3-P-positive events provides evidence that the reagent is effective, even when used in conjunction with lysis solutions that were originally developed to score the incidence of MN. It should be noted that incorporation of an optimized concentration of anti-H3-P reagent affords resolution and scoring of metaphase events without the need for any additional processing steps such as fixation, centrifugation and/or washing. It is also noteworthy that the anti-H3-P reagent is not effective at resolving metaphase and G2 events when it is incorporated into a low pH second lysis solution of the type described herein.

### Example 2

### Treatment with Reference Genotoxicants

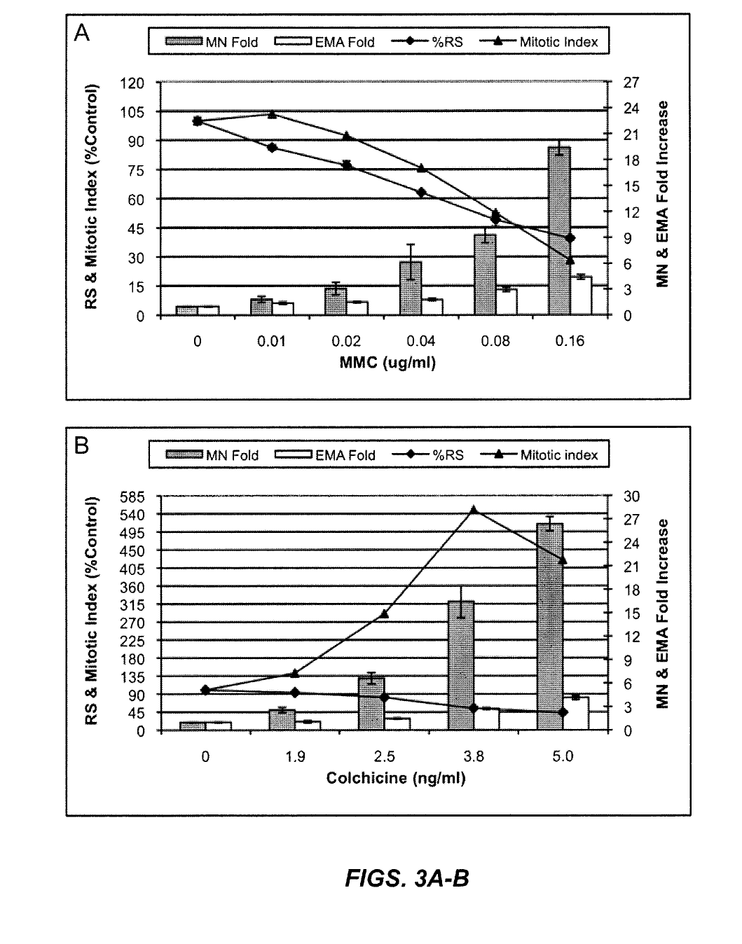

In these experiment, TK6 cells were exposed to a range of genotoxic chemical concentrations in triplicate wells, with a goal of achieving approximately 50% reduction in relative survival at the termination of the experiment (24 to 27 hours after initiation of treatment). The cell cultures contained equal numbers of counting beads which facilitated the relative survival measurements. As described above, at the termination of the treatment period, cells were contacted with a first fluorescent reagent (EMA) in order to label the chromatin associated with dead and/or dying cells. After photoactivaton and washing steps, the cells were brought into simultaneous contact with detergent to liberate nuclei, MN, metaphase chromosomes, and chromatin debris, a second fluorescent reagent (anti-H3-P Alexa 647 conjugate as a metaphase specific fluorochrome), a third fluorescent reagent (SYTOX® Green as a pan-DNA fluorescent dye), and RNase. The liberated nuclei, MN, metaphase chromosomes, and chromatin debris were subsequently contacted with a second lysis solution and were then analyzed on a dual-laser flow cytometer. FIG. 3 shows response profiles of one representative clastogen (MMC) and one representative aneugen (colchicine). As expected, both the clastogen and aneugen show dose-dependent increases in MN frequency, and dose-dependent cytotoxicity as evidence by decreased % RS values and increased % EMA values. These endpoints alone are therefore not capable of discriminating between clastogenic and aneugenic modes of action. On the other hand, their very different metaphase responses are observed to differentiate these MOA. Specifically, whereas the proportion of metaphase events relative to negative control (referred to as “mitotic index” in FIG. 3) is reduced by increasing concentrations of the clastogen MMC, this parameter is increased by increasing concentrations of the aneugen colchicine. Table II describes the results of 18 reference genotoxicants, and the behavior of micronuclei, relative survival, EMA, and metaphase events follow this same general pattern.

TABLE II
Flow Cytometric Results For 18 Reference Genotoxicants

| | | | | | | Avg. M |
|---|---|---|---|---|---|---|
| Chemical | Conc. | Avg. | Avg. | Avg. | Avg. | as % |
| (MOA) | (μg/ml) | % MN | % RS | % EMA+ | % M | G2/M |
| MMS (C) | 0 | 0.6 | 100.0 | 2.4 | 1.9 | 6.8 |
| | 1.5 | 1.3 | 79.6 | 4.0 | 1.0 | 2.1 |
| | 2.3 | 1.6 | 69.5 | 4.7 | 0.9 | 1.8 |
| | 3.0 | 2.2 | 63.4 | 5.5 | 1.0 | 1.6 |
| | 4.5 | 3.6 | 54.2 | 7.5 | 1.8 | 2.1 |
| | 6.0 | 4.3 | 49.4 | 8.3 | 0.7 | 1.2 |
| HU (C) | 0 | 0.4 | 100.0 | 2.4 | 1.8 | 5.2 |
| | 0.5 | 0.4 | 100.9 | 2.4 | 1.6 | 5.4 |
| | 0.9 | 0.5 | 97.1 | 2.3 | 1.6 | 5.9 |
| | 1.9 | 0.6 | 84.3 | 3.5 | 2.0 | 6.7 |
| | 3.8 | 0.9 | 65.7 | 5.8 | 1.7 | 4.7 |
| | 7.5 | 1.9 | 44.3 | 13.7 | 1.0 | 3.9 |
| Etoposide (C) | 0 | 0.7 | 100.0 | 2.3 | 1.7 | 6.4 |
| | 0.0025 | 1.3 | 89.3 | 2.3 | 1.7 | 5.9 |
| | 0.005 | 2.1 | 83.3 | 2.3 | 1.6 | 5.7 |
| | 0.01 | 3.6 | 69.6 | 3.7 | 1.4 | 5.2 |
| | 0.02 | 6.0 | 52.6 | 5.3 | 1.2 | 3.8 |
| MMC (C) | 0 | 0.8 | 100.0 | 1.8 | 1.9 | 6.3 |
| | 0.01 | 1.4 | 86.2 | 2.5 | 2.0 | 5.4 |
| | 0.02 | 2.3 | 77.1 | 2.7 | 1.8 | 4.6 |
| | 0.04 | 4.6 | 63.2 | 3.2 | 1.5 | 3.2 |
| | 0.08 | 7.0 | 49.1 | 5.3 | 1.0 | 1.9 |
| Ara-C (C) | 0 | 0.6 | 100.0 | 3.5 | 1.9 | 6.1 |
| | 1.3 | 0.7 | 82.4 | 3.6 | 2.1 | 5.8 |
| | 2.5 | 1.2 | 73.8 | 4.1 | 2.2 | 6.3 |
| | 5.0 | 1.6 | 65.1 | 4.7 | 1.6 | 5.0 |
| | 10.0 | 2.5 | 57.0 | 6.3 | 1.4 | 4.5 |
| | 20.0 | 5.4 | 45.2 | 10.2 | 1.2 | 3.7 |
| 5-FU (C) | 0 | 0.6 | 100.0 | 3.1 | 1.8 | 6.0 |
| | 0.2 | 0.7 | 96.8 | 3.5 | 1.6 | 5.9 |
| | 0.3 | 0.9 | 90.6 | 4.0 | 1.5 | 6.5 |
| | 0.6 | 2.4 | 79.9 | 5.1 | 1.3 | 6.2 |
| | 1.3 | 5.4 | 62.4 | 7.8 | 0.8 | 5.1 |
| | 2.5 | 6.2 | 48.9 | 12.1 | 0.5 | 4.5 |
| Bleomycin (C) | 0 | 1.4 | 100.0 | 3.0 | 1.9 | 5.2 |
| | 0.05 | 2.7 | 81.9 | 5.1 | 1.7 | 5.6 |
| | 0.1 | 3.4 | 77.7 | 5.6 | 1.7 | 4.9 |
| | 0.2 | 3.9 | 71.3 | 7.7 | 1.6 | 4.6 |
| | 0.4 | 4.9 | 67.2 | 7.3 | 1.5 | 4.4 |
| | 0.8 | 7.1 | 53.5 | 10.9 | 1.3 | 3.8 |
| Camptothecin | 0 | 0.8 | 100.0 | 3.8 | 1.7 | 6.1 |
| (C) | 0.0625 | 0.8 | 98.5 | 3.7 | 1.7 | 5.5 |
| | 0.125 | 0.8 | 88.9 | 5.7 | 1.6 | 5.7 |
| | 0.25 | 1.2 | 79.4 | 6.4 | 1.8 | 6.1 |
| | 0.5 | 1.5 | 76.0 | 5.2 | 2.2 | 6.7 |
| | 1.0 | 5.4 | 49.2 | 10.7 | 1.8 | 4.4 |
| Aphidicolin | 0 | 0.8 | 100.0 | 2.2 | 1.8 | 5.1 |
| (C) | 0.0025 | 0.9 | 97.4 | 2.2 | 1.7 | 4.7 |
| | 0.005 | 0.9 | 88.6 | 2.4 | 1.8 | 5.1 |
| | 0.01 | 1.1 | 78.4 | 2.6 | 1.9 | 5.8 |
| | 0.02 | 2.1 | 65.5 | 2.9 | 1.6 | 4.5 |
| | 0.04 | 5.3 | 54.7 | 4.1 | 1.1 | 4.4 |
| MNNG (C) | 0 | 0.6 | 100.0 | 2.8 | 1.8 | 6.0 |
| | 0.003125 | 2.0 | 78.3 | 4.3 | 1.0 | 1.9 |
| | 0.00625 | 4.4 | 69.7 | 5.9 | 0.6 | 1.1 |
| | 0.0125 | 8.9 | 59.1 | 7.3 | 0.4 | 0.9 |
| | 0.025 | 8.3 | 48.6 | 10.3 | 0.4 | 0.9 |
| | 0.05 | 9.1 | 44.1 | 11.0 | 0.4 | 1.1 |
| Vinblastine | 0 | 0.9 | 100.0 | 1.9 | 1.7 | 6.3 |
| sulfate (A) | 0.1 | 0.7 | 88.0 | 2.0 | 1.8 | 8.6 |
| | 0.2 | 1.6 | 79.6 | 2.7 | 1.5 | 9.0 |
| | 0.4 | 3.3 | 70.6 | 4.0 | 2.7 | 17.1 |
| | 0.8 | 7.5 | 48.0 | 5.8 | 7.2 | 32.0 |
| | 1.6 | 12.6 | 37.6 | 8.8 | 4.4 | 14.1 |
| Carbendazim | 0 | 0.8 | 100.0 | 2.3 | 1.8 | 6.4 |
| (A) | 0.125 | 0.8 | 96.9 | 2.3 | 1.6 | 6.1 |
| | 0.25 | 1.1 | 90.2 | 3.2 | 1.5 | 6.5 |
| | 0.5 | 3.8 | 71.2 | 6.4 | 1.6 | 10.3 |
| | 1 | 14.7 | 49.7 | 10.0 | 5.6 | 24.9 |
| | 2 | 22.3 | 43.3 | 12.0 | 5.4 | 18.5 |
| Colchicine | 0 | 0.8 | 100.0 | 2.7 | 1.8 | 6.2 |
| (A) | 1.9 | 2.1 | 93.5 | 2.9 | 2.5 | 10.5 |
| | 2.5 | 5.4 | 81.3 | 4.0 | 5.1 | 21.9 |
| | 3.8 | 13.4 | 54.2 | 7.4 | 9.6 | 36.3 |
| | 5.0 | 21.6 | 43.6 | 11.1 | 7.4 | 19.8 |
| Vincristine | 0 | 0.7 | 100.0 | 3.2 | 2.0 | 6.5 |
| sulfate (A) | 0.125 | 0.6 | 91.9 | 3.3 | 1.9 | 7.1 |
| | 0.25 | 0.9 | 86.2 | 3.4 | 1.8 | 7.2 |
| | 0.5 | 2.7 | 72.1 | 5.1 | 2.1 | 8.4 |
| | 1.0 | 7.9 | 53.1 | 8.2 | 3.4 | 13.5 |
| Paclitaxel | 0 | 0.7 | 100.0 | 2.3 | 1.8 | 6.4 |
| (A) | 1.6 | 1.5 | 90.1 | 3.7 | 1.7 | 7.1 |
| | 2.4 | 4.4 | 71.8 | 7.3 | 2.2 | 9.7 |
| | 3.3 | 9.2 | 60.8 | 10.0 | 3.4 | 12.2 |
| | 4.9 | 12.5 | 52.2 | 12.3 | 4.4 | 11.3 |
| | 6.5 | 15.8 | 50.6 | 13.2 | 4.6 | 10.2 |
| | 0 | 0.7 | 100.0 | 2.6 | 1.9 | 6.2 |
| | 0.375 | 0.7 | 96.6 | 3.0 | 1.9 | 6.0 |
| Griseofulvin | 0.75 | 0.7 | 99.5 | 2.6 | 1.8 | 5.8 |
| (A) | 1.5 | 1.3 | 90.0 | 3.6 | 1.6 | 7.5 |
| | 3.0 | 6.1 | 64.9 | 8.2 | 3.0 | 19.4 |
| | 6.0 | 14.8 | 46.6 | 13.5 | 5.8 | 26.4 |
| DES (A) | 0 | 0.6 | 100.0 | 2.7 | 1.9 | 7.1 |
| | 1.3 | 0.8 | 86.3 | 3.1 | 1.8 | 6.6 |
| | 1.9 | 1.1 | 82.1 | 4.0 | 1.8 | 8.0 |
| | 2.5 | 2.3 | 65.6 | 6.2 | 2.3 | 12.7 |
| | 3.8 | 6.1 | 54.8 | 7.5 | 4.9 | 22.9 |
| | 5.0 | 9.9 | 40.5 | 13.0 | 6.8 | 29.3 |

% MN = percent micronuclei; % RS = percent relative survival; % EMA+ = percent ethidium monoazide positive events; % M = percent metaphase events; M as % G2/M = percentage of metaphase events relative to G2 and metaphase events.

### Example 3

### Polyploidy

In this experiment, TK6 cells were treated with solvent, the clastogen cisplatin, or the aneugen paclitaxel. Treatments occurred in 96 well plates, demonstrating the ability to scale the invention to smaller vessels and thereby reduce the amount of test article required for testing. As described above, at the termination of the treatment period, cells were contacted with a first fluorescent reagent (EMA) in order to label the chromatin associated with dead and/or dying cells. After photoactivaton and washing steps, the cells were brought into simultaneous contact with detergent to liberate nuclei, MN, metaphase chromosomes, and chromatin debris, a second fluorescent reagent (anti-H3-P Alexa 647 conjugate as a metaphase specific fluorochrome), a third fluorescent reagent (SYTOX® Green as a pan-DNA fluorescent dye), and RNase. The liberated nuclei, MN, metaphase chromosomes, and chromatin debris were subsequently contacted with a second lysis solution and were then analyzed on a dual-laser flow cytometer. FIG. 6 shows the robust induction of polyploid nuclei (8n) for the aneugen paclitaxel, but not the clastogen cisplatin.

### Discussion of Examples 1-3

The methodology described herein has been found to provide new levels of efficiency and information content to the in vitro micronucleus assay. It simultaneously and comprehensively evaluates genotoxicity and cytotoxicity, and in the case of genotoxicity provides valuable MOA information. Current practices and state-of-the-art have not successfully multiplexed these evaluations as completely or in as quantitative of a manner as the present invention does.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method for the enumeration of eukaryotic cell micronuclei, while simultaneously acquiring data characterizing cytotoxicity and for distinguishing between aneugenic and clastogenic modes of action, the method comprising:
contacting a sample containing eukaryotic cells with a first fluorescent reagent that permeates dead and dying cells but not viable cells, that covalently binds chromatin, and that has a fluorescence emission spectrum;
contacting the sample with one or more lysis solutions that result in digestion of eukaryotic cell outer membranes but retention of nuclear membranes, thereby forming free nuclei, micronuclei, bundles of metaphase chromosomes, chromatin debris from dead and/or dying cells, or any combinations thereof;
contacting the free nuclei and/or micronuclei and/or metaphase chromosomes and/or chromatin debris with RNase to substantially degrade RNA;
contacting metaphase chromosomes with a second fluorescent reagent that binds to metaphase chromosome-associated epitopes and whose fluorescence emission spectrum does not substantially overlap with the fluorescent emission spectrum of the first fluorescent reagent;
staining cellular DNA with a third fluorescent reagent having a fluorescent emission spectrum that does not substantially overlap with the fluorescent emission spectrum of the first or second fluorescent reagents;
exciting the first, second, and third fluorescent reagents with light of appropriate excitation wavelength(s); and
detecting the fluorescent emission and light scatter produced by the nuclei and/or micronuclei and/or metaphase chromosomes and/or chromatin debris, and counting (i) the number of micronuclei in said sample relative to the number of nuclei, or (ii) the number of events that exhibit metaphase-specific fluorescence relative to the number of nuclei and/or relative to G2/M nuclei, or (iii) the number of chromatin debris events relative to the number of nuclei, or (iv) the number of polyploid nuclei relative to the number of nuclei, or any combination of (i) to (iv) to characterize cytotoxicity and distinguish between aneugenic and clastogenic modes of action.

2. A method for the enumeration of eukaryotic cell micronuclei, while simultaneously acquiring data characterizing cytotoxicity and genotoxicity, and for distinguishing between aneugenic and clastogenic modes of action, the method comprising:
exposing a eukaryotic cell sample comprising whole cells and dead and dying cells to (i) first, second, and third fluorescent reagents that are characterized by fluorescent emission spectra that do not substantially overlap, and (ii) a lysis solution that lyses cellular membranes, said exposing being carried out under conditions effective to allow the first fluorescent reagent to label chromatin debris of dead and dying cells, the second fluorescent reagent to label metaphase chromosome-associated epitopes, and the third fluorescent reagent to label all cellular DNA, including chromatin debris, metaphase chromosomes, nuclei, and micronuclei;
exciting the first, second, and third fluorescent reagents; and
detecting the fluorescent emission and light scatter produced by the nuclei and/or micronuclei and/or metaphase chromosomes and/or chromatin debris, and counting (i) the number of micronuclei in said sample relative to the number of nuclei, or (ii) the number of metaphase events relative to the number of nuclei and/or G2/M nuclei, (iii) the number of chromatin debris events relative to the number of nuclei, or (iv) the number of polyploid nuclei relative to the number of nuclei, or any combination thereof, to characterize cytotoxicity and genotoxicity, and in the case of genotoxicity, to distinguish between aneugenic and clastogenic modes of action.

3. A method for assessing cytotoxicity or genotoxicity of a chemical or physical agent, and distinguishing between aneugenic and clastogenic modes of action, the method comprising:
exposing a eukaryotic cell sample, previously exposed to a chemical or physical agent and comprising whole cells and dead and/or dying cells, to (i) first, second, and third fluorescent reagents that are characterized by fluorescent emission spectra that do not substantially overlap, and (ii) a lysis solution that lyses cellular membranes, said exposing being carried out under conditions effective to allow the first fluorescent reagent to label chromatin debris, the second fluorescent reagent to label metaphase events, and the third fluorescent reagent to label all cellular DNA, including chromatin debris, metaphase events, nuclei, and micronuclei, and (iii) a known concentration of counting beads;
exciting the first, second, and third fluorescent reagents; and
detecting the fluorescent emission and light scatter produced by the nuclei and/or micronuclei and/or metaphase chromosomes, and/or chromatin debris, and counting beads, and determining one or more of the following endpoints: (i) the frequency of first fluorescent reagent-positive events, as a measure of cytotoxicity; (ii) the ratio of third fluorescent reagent-positive and first fluorescent reagent-negative nuclei to counting beads as a measure of cytotoxicity; (iii) the frequency of second and third fluorescent reagent-positive and first fluorescent reagent-negative metaphase chromosomes relative to third fluorescent-positive and first fluorescent reagent-negative nuclei and/or relative to total third fluorescent-positive and first fluorescent reagent-negative G2/M nuclei, whereby decrease(s) relative to a baseline value or negative control represents a measure of cytotoxicity and increase(s) relative to a baseline value or negative control represents an indication of an aneugenic mode of genotoxic activity; (iv) the frequency of third fluorescent reagent-positive and first fluorescent reagent-negative polyploidy nuclei relative to total third fluorescent-positive and first fluorescent reagent-negative nuclei, whereby an increase relative to a baseline value or negative control indicates an aneugenic mode of genotoxic activity; and (v) the proportion of third fluorescent reagent-positive and first fluorescent reagent-negative micronuclei relative to third fluorescent reagent-positive and first fluorescent reagent-negative nuclei as a measure of genotoxicity.

4. The method according to claim 1, wherein the first fluorescent reagent is a DNA dye that is in an inactive form during said contacting or said exposing, the method further comprising:
photoactivating the DNA dye into a reactive form that covalently binds chromatin.

5. The method according to claim 4 wherein the first fluorescent DNA dye is ethidium monoazide bromide and/or propidium monoazide bromide.

6. The method according to claim 1, wherein the third fluorescent reagent is a nucleic acid dye.

7. The method according to claim 1, wherein the second fluorescent reagent is a fluorochrome-conjugated anti-phosphorylated histone H3 antibody.

8. The method according to claim 1, wherein the one or more lysis solutions comprises a first lysis solution comprising NaCl, Na-Citrate, and octylphenyl-polyethylene glycol in deionized water; and a second lysis solution comprises citric acid and sucrose in deionized water.

9. The method according to claim 1 wherein said contacting with one or more lysis solutions and said contacting with RNase are carried out simultaneously.

10. The method according to claim 1 wherein said contacting with one or more lysis solutions and said contacting with RNase are carried out sequentially.

11. The method according to claim 1 wherein said contacting with one or more lysis solutions, said contacting with RNase, contacting metaphase chromosomes with the second fluorescent reagent, and said staining cellular DNA with the third fluorescent reagent are carried out simultaneously.

12. The method according to claim 1 wherein said contacting with one or more lysis solutions, said contacting with RNase, contacting metaphase chromosomes with the second fluorescent reagent, and said staining cellular DNA with the third fluorescent reagent are carried out sequentially.

13. The method according to claim 1 wherein the eukaryotic cells are cultured in vitro.

14. The method according to claim 1, further comprising treating the eukaryotic cell sample with a chemical or physical agent prior to any of said contacting steps or said exposing.

15. The method according to claim 14, wherein the chemical or physical agent causes genetic and/or cytotoxic damage, the method further comprising:
treating the eukaryotic cell sample with a second agent that may modify genetic or cytotoxic damage caused by the chemical or physical agent.

16. The method according to claim 1 further comprising:
calculating the frequency of micronuclei relative to total detected nuclei, and/or the frequency of metaphase events relative to total detected nuclei or G2/M events.

17. The method according to claim 1, wherein said exciting is carried out with a single-laser or multiple-laser flow cytometer.

18. A method of assessing the DNA-damaging potential of a chemical or physical agent comprising:
exposing eukaryotic cells to a chemical or physical agent, and
performing the method according to claim 1, wherein a significant increase in the frequency of micronuclei from a baseline micronuclei value in unexposed or negative control eukaryotic cells indicates the genotoxic potential of the chemical or physical agent, a significant decrease in the number of events that exhibit metaphase-specific fluorescence relative to number of nuclei and/or G2/M nuclei indicates the cytotoxic potential of the chemical or physical agent, a significant elevation in the frequency of metaphase-positive events relative to number of nuclei and/or G2/M nuclei from a baseline value in unexposed or negative control eukaryotic cells indicates genotoxicity with an aneugenic mode of action, and a significant elevation in the frequency of polyploidy nuclei relative to the number of nuclei from a baseline value in unexposed or negative control eukaryotic cells indicates genotoxicity with an aneugenic mode of action.

19. The method according to claim 18, wherein said exposing is carried out for a predetermined period of exposure time.

20. A method of assessing the cytotoxicity of a chemical or physical agent, said method comprising;
exposing eukaryotic cells to a chemical or physical agent and
performing the method according to claim 1, wherein a significant increase in the frequency of chromatin debris relative to nuclei events from a baseline value in unexposed or negative control eukaryotic cells indicates the cytotoxic potential of the chemical or physical agent, and/or a significant decrease in the number of metaphase events relative to number of nuclei and/or G2/M nuclei indicates the cytotoxic potential of the chemical or physical agent, and/or a significant decrease in the proportion of nuclei to counting beads, relative to a baseline value in unexposed or negative control eukaryotic cells, indicates the cytotoxic potential of the chemical or physical agent.

21. The method according to claim 20, wherein said exposing is carried out for a predetermined period of exposure time.

22. A kit comprising:
one or more eukaryotic cell membrane lysis solutions;
a first fluorescent reagent that permeates the dead and dying cells, but not viable cells;
a second fluorescent reagent that specifically labels metaphase chromosome-associated epitopes, wherein the second fluorescent reagent has a fluorescent emission spectrum that does not substantially overlap with a fluorescent emission spectrum of the first fluorescent reagent;
a third fluorescent reagent that labels all chromatin having a fluorescent emission spectrum, wherein the third fluorescent reagent has a fluorescent emission spectrum that does not substantially overlap with a fluorescent emission spectrum of the first and second fluorescent reagents; and
RNase A solution.

23. The kit according to claim 22, wherein the first fluorescent reagent is ethidium monoazide bromide and/or propidium monoazide bromide.

24. The kit according to claim 22, wherein the second fluorescent reagent is a fluorochrome-conjugated anti-phosphorylated histone H3 antibody.

25. The kit according to claim 22, wherein the third fluorescent reagent is a pan-nucleic acid dye.

26. The kit according to claim 22 further comprising one or more of:
counting beads;
instructions that describe cell harvest and staining procedures, and also scoring of micronuclei, nuclei, G2/M nuclei, chromatin from dead and dying cells, and metaphase-specific chromosomes;
a computer readable storage medium that contains a cytometry data acquisition template for flow cytometric scoring of micronuclei, nuclei, G2/M nuclei, chromatin from dead and dying cells, and metaphase-specific chromosomes; and
a container comprising an in vitro culture of eukaryotic cells.
